(19) United States
(12) Patent Application Publication
(10) Pub. No.: US 2020/0216563 A1
(43) Pub. Date: Jul. 9, 2020

(54) HDAC6 AND PROTEIN AGGREGATION

(71) Applicant: Friedrich Miescher Institute for Biomedical Research, Basel (CH)

(72) Inventors: Patrick Daniel Matthias, Basel (CH); Makoto Saito, Tokyo (JP)

(21) Appl. No.: 16/637,569

(22) PCT Filed: Aug. 8, 2018
(86) PCT No.: PCT/IB2018/055969

§ 371 Date: Feb. 7, 2020

(30) Foreign Application Priority Data
Aug. 10, 2017 (EP) 17185730.3

(51) Int. Cl. C07K 16/40

(52) U.S. Cl. CPC C07K 16/40; C07K 2317/76

(57) ABSTRACT

The present application provides a HDAC6 inhibitor for use in treating a degenerative disease associated with the formation of intracellular granules, wherein said granules are formed by the aggregation of a protein selected from the group comprising APP, Tau, Ataxin-2, hnRNPA1, C9orf72, Lamin A/C, Myotilin, Matrin 3, alpha-synuclein, SOD1, FUS, hnRNPA2B1, VCP, TIA1, Desmin and Huntingtin, and wherein said inhibitor inhibits both the CD1 and CD2 of HDAC6.

### FIELD OF THE INVENTION

The present invention provides a method in treating a degenerative disease associated with the formation of intracellular granules.

### BACKGROUND OF THE INVENTION

In subjects having neurodegenerative disease neurons of the brain and spinal cord are lost. Examples of neurodegenerative diseases include Alexander disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease (Spinocerebellar ataxia type 3), Multiple sclerosis, Multiple System Atrophy, Neuroborreliosis, Parkinson disease, Pelizaeus-Merzbacher Disease, Pick's disease, Primary lateral sclerosis, Prion diseases, Refsum's disease, Sandhoff disease, Schilder's disease, Sub-Acute Combined Degeneration of the Cord Secondary to Pernicious Anaemia, Schizophrenia, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Tabes dorsalis and Charcot-Marie-Tooth disease.

Neurodegenerative pathologies represent a major class of human diseases, despite intensive research a complete picture of the underlying mechanisms is still lacking and there is a need for effective treatments for neurodegeneration. Many of these diseases share the feature of accumulation/aggregation of mutated, abnormally folded proteins in neuronal cell types, e.g. of Amyloid beta and tau proteins in the cerebral cortex in Alzheimer's disease, alpha-synuclein in the substantia nigra in Parkinson's disease, Huntingtin in the striatum and other brain areas in Huntington's disease (HD) or Superoxide Dismutase 1 (SOD1) in motoneurons in familial Amyotrophic Lateral Sclerosis (fALS). Cellular death is the consequence of the pathological processes, but it is under debate whether and in which cases protein aggregation is a direct effect of disease progression and toxic for the cells or, conversely, is part of cellular defense systems and has therefore to be considered cytoprotective (Lee, H. G., X. Zhu, et al. (2006). Exp Neurol 200(1): 1-7.). The cytosolic deacetylase HDAC6 functions at the crossroads of stress responses, formation of protein aggregates and their autophagic clearance. The enzyme deacetylates the chaperone HSP90 and thereby controls the function of its client proteins, as has been shown for Glucocorticoid receptor activation (Kovacs, J. J., P. J. Murphy, et al. (2005) Mol Cell 18(5): 601-7). Besides deacetylation, HDAC6 binds ubiquitin with very high affinity (Boyault, C., B. Gilquin, et al. (2006). Embo J 25(14): 3357-66) and acts as a stress sensor by recognizing an excess of ubiquitinated proteins and thereupon initiating heat shock gene activation via induction of the heat shock transcription factor Hsf1 (Boyault, C., Y. Zhang, et al. (2007). Genes Dev 21(17): 2172-81.). Alpha-tubulin is another deacetylation target of HDAC6, and HDAC6 colocalizes with microtubule-associated dynein motor proteins (Hubbert, C., A. Guardiola, et al. (2002). Nature 417(6887): 455-8; Zhang et al., EMBO 2003). HDAC6 regulates microtubule dynamics (Zilberman, Y., C. Ballestrem, et al. (2009). J Cell Sci 122(Pt 19): 3531-41.), cell motility (Zhang, X., Z. Yuan, et al. (2007). Mol Cell 27(2): 197-213), axonal transport (d'Ydewalle, C., J. Krishnan, et al. Nat Med 17(8): 968-74) and microtubule-based transfer of ubiquitinated proteins to the aggresome in response to stress (Kawaguchi, Y., J. J. Kovacs, et al. (2003). Cell 115(6): 727-38). In addition, HDAC6 is required for stress granule formation (Kwon, S., Y. Zhang, et al. (2007). Genes Dev 21(24): 3381-94). Furthermore, HDAC6 is directly involved in autophagic clearance of protein aggregates by deacetylating the actin-binding protein cortactin and thereby regulates the fusion of autophagosomes with lysosomes (Lee, J. Y., H. Koga, et al. Embo J 29(5): 969-80).

However, the difficulty to translate observations from biochemical and cellular studies into models explaining disease progression in complex higher organisms is highlighted by the finding that despite promising cell culture results in various systems, no impact of HDAC6 on disease progression was observed in a transgenic mouse model of Huntington's disease (Bobrowska, A., P. Paganetti, et al. PLoS One 6(6): e20696).

This difficulty is further aggravated by the fact that HDAC6 possesses two different domains with deacetylase activity (DD1 and DD2, also termed CD1 and CD2), the deacetylase activity differing between the two domains, wherein only DD2 is considered to have a biologically relevant deacetylase activity (Zou et al., 2006, Biochemical and Biophysical Research Communications, 341; 45-50; Boyault et al., 2007, Oncogene, 26:5468-5476; Haggarty et al., 2003, PNAS, 100(8), 4389-4394).

### SUMMARY OF THE INVENTION

The present inventors now surprisingly found that the removal of acetyl groups in intrinsically disordered regions (IDRs) of certain proteins by both the CD1 and CD2 HDAC6 lead to the aggregation of said protein and maturation of stress granules. The inventors show that deacetylation of the IDR by HDAC6 affects its capacity to undergo liquid-liquid phase separation in vitro, a property that underlies protein aggregation. The present invention thus provides a HDAC6 inhibitor for use in treating a degenerative disease associated with the formation of intracellular granules, wherein said granules are formed by the aggregation of a protein selected from the group comprising APP, Tau, Atx2, hnRNPA1, C9orf72, Lamin NC, Myitilin, Matrin 3alpha-synuclein, SOD1, FU, hnRNPA2, VCP, TIA1, Desmin and Hungtitin, wherein said inhibitor inhibits both the CD1 and CD2 of HDAC6. The present invention also provides a method for treating, in a patient in need thereof, a degenerative disease associated with the formation of intracellular granules, wherein said granules are formed by the aggregation of a protein selected from the group comprising APP, Tau, Atx2, hnRNPA1, C9orf72, Lamin NC, Myitilin, Matrin 3alpha-synuclein, SOD1, FU, hnRNPA2, VCP, TIA1, Desmin and Hungtitin, said method comprising administering a therapeutically effective amount of an inhibitor inhibiting both the CD1 and CD2 of HDAC6.

### DESCRIPTION OF THE FIGURE

FIG. 1: Lysine Acetylation in IDRs affects precipitation in vitro and is regulated by either HDAC6 catalytic domain. (A) Acetylation of the DDX3X IDR regulates its ability to precipitate in vitro. Left: lysates from 293T cells expressing GFP (control) or CBP were subjected to B-isox-mediated precipitation and analyzed by immunoblotting. The presence of endogenous DDX3X-K118Ac was assessed in the precipitate fraction (B-isox ppt), as well as in the lysate fraction (input) and compared to the level of total precipitated DDX3X. TIAR was used as a control protein harboring an IDR, while α-tubulin and ubiquitin were used as control proteins lacking an IDR. Proteins were detected with specific antibodies, as indicated. Right: schematic illustrating the B-isox-mediated precipitation of non-acetylated IDRs, while acetylated IDRs remain in suspension. (B) Almost all of the 18 high-confidence HDAC6 sites map to IDRs. Left panel: PONDR analysis was performed with the 18 high-confidence HDAC6 sites as well as with all (2029) acetylated sites examined here, as control. Right panel: HDAC6 KO 293T cellular lysates were used for a B-isox-mediated precipitation. Proteins were detected by immunoblotting with specific antibodies, as indicated. (C) DDX3X and cortactin can be deacetylated by either HDAC6 catalytic domain. Left, top: schematic drawing of HDAC6 structure highlighting the tandem catalytic domains (CD1, CD2) as well as the zinc finger domain binding ubiquitin (ZnF). The positions of the different mutations are indicated. Left, bottom: deacetylation of DDX3X, cortactin and alpha-tubulin by HDAC6 and its functional mutants. Lysates from HDAC6 KO 293T cells expressing CBP (lanes 1-6) and the different HDAC6 constructs (WT, H216A, H611A, H216A/H611A, or W1182A, lanes 2 to 6) were used to detect the acetylation status of the different proteins by immunoblotting. For DDX3X and alpha-tubulin, antibodies specific for the respective acetylated site were used (Ac-DDX3X-K118 and Ac-alpha-tubulin-K40) were used; for cortactin a pan-acetylated-lysine antibody was used. Right: PONDR analysis of DDX3X, cortactin and alpha-tubulin illustrating the position of the acetylated sites with respect to IDRs or ordered regions.

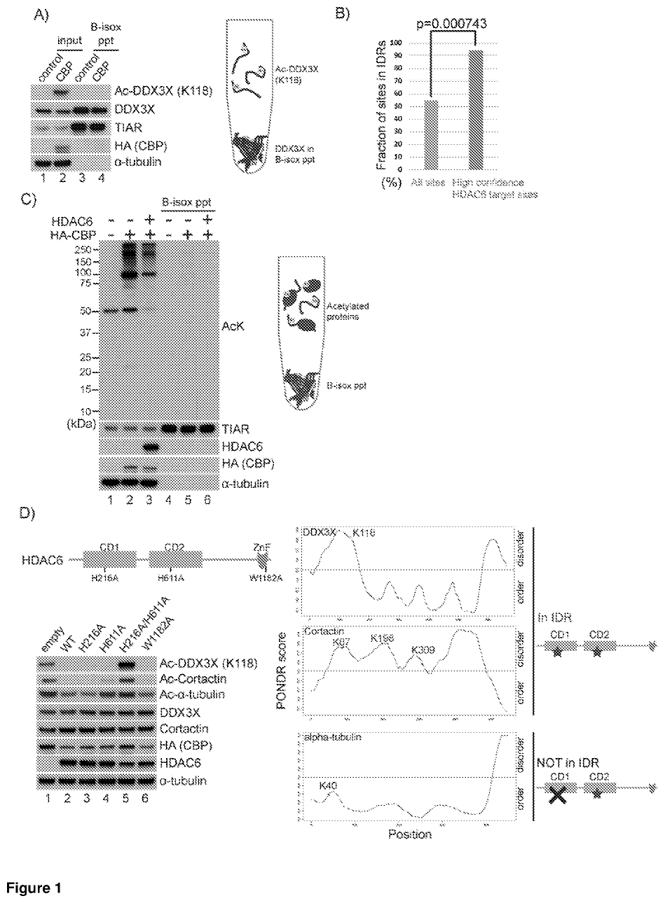

### DETAILED DESCRIPTION OF THE INVENTION

The present inventors surprisingly found that the removal of acetyl groups in mostly disordered regions of certain proteins by both the CD1 and CD2 HDAC6 lead to the aggregation of said protein and maturation of stress granules. They found that removal of acetylation from the IDR by HDAC6 promoted the liquid-liquid phase separation of the IDR, which underlies its capacity to aggregate in vitro; in agreement with this, HDAC6 deacetylation also favored formation of mature large stress granules in vivo.

The present invention thus provides a HDAC6 inhibitor for use in treating a degenerative disease associated with the formation of intracellular granules, wherein said granules are formed by the aggregation of a protein selected from the group comprising APP, Tau, Ataxin-2, hnRNPA1, C9orf72, Lamin NC, Myotilin, Matrin 3, alpha-synuclein, SOD1, FUS, hnRNPA2B1, VCP, TIA1, Desmin and Huntingtin, wherein said inhibitor inhibits both the CD1 and CD2 of HDAC6.

The present invention also provides a method for treating, in a patient in need thereof, a degenerative disease associated with the formation of intracellular granules, wherein said granules are formed by the aggregation of a protein selected from the group comprising APP, Tau, Atx2, hnRNPA1, C9orf72, Lamin NC, Myitilin, Matrin 3alpha-synuclein, SOD1, FU, hnRNPA2, VCP, TIA1, Desmin and Hungtitin, said method comprising administering a therapeutically effective amount of an inhibitor inhibiting both the CD1 and CD2 of HDAC6.

APP, or Amyloid precursor protein, is an integral membrane protein expressed in many tissues and concentrated in the synapses of neurons. Its primary function is not known.APP is best known as the precursor molecule whose proteolysis generates beta amyloid (Aβ), a polypeptide containing 37 to 49 amino acid residues, whose amyloid fibrillar form is the primary component of amyloid plaques found in the brains of Alzheimer's disease patients.

Tau proteins (or τ proteins) are proteins that stabilize microtubules. They are abundant in neurons of the central nervous system, but are also expressed at very low levels in CNS astrocytes and oligodendrocytes. Pathologies and dementias of the nervous system such as Alzheimer's disease and Parkinson's disease are associated with tau proteins that have become defective and no longer stabilize microtubules properly. The tau proteins are the product of alternative splicing from a single gene that in humans is designated MAPT (microtubule-associated protein tau) and is located on chromosome 17.

Ataxin-2 is a protein that in humans is encoded by the ATXN2 gene. Mutations in ATXN2 cause spinocerebellar ataxia type 2 (SCA2). SCA2 is caused by the expansion of a CAG repeat in the coding region of the ATXN2 gene producing an elongated polyglutamine tract in the corresponding protein. The expanded repeats are variable in size and unstable, usually increasing in size when transmitted to successive generations.

hnRNPA1 or heterogeneous nuclear ribonucleoprotein A1 is a protein that in humans is encoded by the HNRNPA1 gene. Mutations in hnRNPA1 are a cause of amyotrophic lateral sclerosis and multisystem proteinopathy.

C9orf72 (chromosome 9 open reading frame 72) is a protein which in humans is encoded by the gene C9orf72. The protein is found in many regions of the brain, in the cytoplasm of neurons as well as in presynaptic terminals. Mutations in C9orf72 are significant because it is the first pathogenic mechanism identified to be a genetic link between familial frontotemporal dementia (FTD) and of amyotrophic lateral sclerosis (ALS).

Lamin NC also known as LMNA is a protein that in humans is encoded by the LMNA gene. Mutations in the LMNA gene are associated with several diseases, including Emery-Dreifuss muscular dystrophy, familial partial lipodystrophy, limb girdle muscular dystrophy, dilated cardiomyopathy, Charcot-Marie-Tooth disease, restrictive dermopathy and Hutchinson-Gilford progeria syndrome.

Myotilin is a protein that in humans is encoded by the MYOT gene. Myotilin (myofibrillar titin-like protein) also known as TTID (TiTin Immunoglobulin Domain) is a muscle protein that is found within the Z-disc of sarcomeres. Myotilin is mutated in various forms of muscular dystrophy: Limb-Girdle Muscular Dystrophy type 1A (LGMD1A), Myofibrillar Myopathy (MFM), Spheroid Body Myopathy and Distal Myopath.

Matrin-3 is a protein that in humans is encoded by the MATR3 gene. Mutations in the Matrin 3 gene are associated with familial amyotrophic lateral sclerosis.

Alpha-synuclein is a protein that is abundant in the human brain. Alpha-synuclein aggregates to form insoluble fibrils in pathological conditions characterized by Lewy bodies, such as Parkinson's disease, dementia with Lewy bodies and multiple system atrophy.

SOD1 or Superoxide dismutase [Cu—Zn] also known as superoxide dismutase 1 is an enzyme that in humans is encoded by the SOD1 gene, located on chromosome 21. It is implicated in apoptosis and amyotrophic lateral sclerosis.

FUS or RNA-binding protein FUS/TLS (Fused in Sarcoma/Translocated in Sarcoma) is a protein that in humans is encoded by the FUS gene. FUS is a significant disease protein in a subgroup of frontotemporal lobar dementias (FTLDs), previously characterized by immunoreactivity of the neuronal inclusions for ubiquitin, but not for TDP-43 or tau with a proportion of the inclusions also containing a-internexin in a further subgroup known as neuronal intermediate filament inclusion disease (NIFID). HNRNPA2B1 or heterogeneous nuclear ribonucleoproteins A2/B1 is a protein that in humans is encoded by the HNRNPA2B1 gene. The mutation p.D290V/302V in hnRNPA2B1 is implicated in dementia, myopathy, PDB, and ALS. Mutations in hnRNPA2B1 and hnRNPA1 cause of amyotrophic lateral sclerosis and multisystem proteinopathy.

VCP, also known as transitional endoplasmic reticulum ATPase (TER ATPase) or valosin-containing protein (VCP) or CDC48 in S. Cerevisiae is an enzyme that in humans is encoded by the VCP gene. Its main function is to segregate protein molecules from large cellular structures such as protein assemblies, organelle membranes and chromatin, and thus facilitate the degradation of released polypeptides by the multi-subunit protease proteasome. Mutations in VCP cause multisystem proteinopathy (MSP), a dominantly inherited, pleiotropic, degenerative disorder of humans that can affect muscle, bone and/or the central nervous system. MSP can manifest clinically as classical amyotrophic lateral sclerosis (ALS), frontotemporal dementia (FTD), inclusion body myopathy (IBM), Paget's disease of bone (PDB), or as a combination of these disorders.

TIA1 is a 3′UTR mRNA binding protein and can bind the 5′TOPsequence of 5′TOP mRNAs. It is associated with programmed cell death (apoptosis) and regulates alternative splicing of the gene encoding the Fas receptor, an apoptosis-promoting protein

Desmin is a protein that in humans is encoded by the DES gene. Desmin is a muscle-specific, type III intermediate filament that integrates the sarcolemma, Z disk, and nuclear membrane in sarcomeres and regulates sarcomere architecture. Desmin-related myofibrillar myopathy (DRM or desminopathy) is a subgroup of the myofibrillar myopathy diseases and is the result of a mutation in the gene that codes for desmin which prevents it from forming protein filaments, and rather, forms aggregates of desmin and other proteins throughout the cell.

The huntingtin gene, also called the HTT or HD (Huntington disease) gene, is the IT15 (“interesting transcript 15”) gene, which codes for a protein called the huntingtin protein. It is variable in its structure, as the many polymorphisms of the gene can lead to variable numbers of glutamine residues present in the protein. In its wild-type (normal) form, it contains 6-35 glutamine residues. However, in individuals affected by Huntington's disease (an autosomal dominant genetic disorder), it contains more than 36 glutamine residues (highest reported repeat length is about 250).

As used herein, the term “population” may be any group of at least two individuals. A population may include, e.g., but is not limited to, a reference population, a population group, a family population, a clinical population, and a same sex population.

As used herein, the term “polymorphism” means any sequence variant present at a frequency of >1% in a population. The sequence variant may be present at a frequency significantly greater than 1% such as 5% or 10% or more. Also, the term may be used to refer to the sequence variation observed in an individual at a polymorphic site. Polymorphisms include nucleotide substitutions, insertions, deletions and microsatellites and may, but need not, result in detectable differences in gene expression or protein function.

As used herein, the term “polynucleotide” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified e.g. for stability or for other reasons.

As used herein, the term “polypeptide” means any polypeptide comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well-known in the art. Such modifications are well described in basic texts and in more detailed monographs, as well as in a voluminous research literature.

As used herein, the term “reference standard population” means a population characterized by one or more biological characteristics, e.g., drug responsiveness, genotype, haplotype, phenotype, etc.

As used herein, the term “subject” means that preferably the subject is a mammal, such as a human, but can also be an animal, including but not limited to, domestic animals (e.g., dogs, cats and the like), farm animals (e.g., cows, sheep, pigs, horses and the like) and laboratory animals (e.g., monkeys such as cynmologous monkeys, rats, mice, guinea pigs and the like).

As used herein, a “test sample” means a biological sample obtained from a subject of interest. For example, a test sample can be a biological fluid (e.g., serum), cell sample, or tissue, or isolated nucleic acid or polypeptide derived therefrom.

As used herein, the expression “body fluid” is a biological fluid selected from a group comprising blood, bile, blood plasma, serum, aqueous humor, amniotic fluid, cerebrospinal fluid, sebum, intestinal juice, semen, sputum, sweat and urine.

As used herein, the term “dysregulation” means a change that is larger or equal to 1.2 fold and statistically significant (p<0.05, Student's t-test) from the control. For example, a 1.5, 2, 2.5, 3, 3.5, 4, 4.5, or 5 fold change.

As used herein, the term “statistically significant” means a p value <0.05 as compared to the control using the Student's t-test.

The phrase “hybridising specifically to” as used herein refers to the binding, duplexing, or hybridising of an oligonucleotide probe preferentially to a particular target nucleotide sequence under stringent conditions when that sequence is present in a complex mixture (such as total cellular DNA or RNA). Preferably a probe may bind, duplex or hybridise only to the particular target molecule.

The term “stringent conditions” refers to conditions under which a probe will hybridise to its target subsequence, but minimally to other sequences. Preferably a probe may hybridise to no sequences other than its target under stringent conditions. Stringent conditions are sequence-dependent and will be different in different circumstances. Longer sequences hybridise specifically at higher temperatures.

In general, stringent conditions may be selected to be about 5° C. lower than the thermal melting point (Tm) for the specific sequence at a defined ionic strength and pH. The Tm is the temperature (under defined ionic strength, pH, and nucleic acid concentration) at which 50% of the oligonucleotide probes complementary to a target nucleic acid hybridise to the target nucleic acid at equilibrium. As the target nucleic acids will generally be present in excess, at Tm, 50% of the probes are occupied at equilibrium. By way of example, stringent conditions will be those in which the salt concentration is at least about 0.01 to 1.0 M Na⁺ ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

Oligonucleotide probes may be used to detect complementary nucleic acid sequences (i.e., nucleic acid targets) in a suitable representative sample. Such complementary binding forms the basis of most techniques in which oligonucleotides may be used to detect, and thereby allow comparison of, expression of particular genes. Preferred technologies permit the parallel quantitation of the expression of multiple genes and include technologies where amplification and quantitation of species are coupled in real-time, such as the quantitative reverse transcription PCR technologies and technologies where quantitation of amplified species occurs subsequent to amplification, such as array technologies.

Array technologies involve the hybridisation of samples, representative of gene expression within the subject or control sample, with a plurality of oligonucleotide probes wherein each probe preferentially hybridises to a disclosed gene or genes. Array technologies provide for the unique identification of specific oligonucleotide sequences, for example by their physical position (e.g., a grid in a two-dimensional array as commercially provided by Affymetrix Inc.) or by association with another feature (e.g. labelled beads as commercially provided by Illumina Inc or Luminex Inc). Oligonuleotide arrays may be synthesised in situ (e.g by light directed synthesis as commercially provided by Affymetrix Inc) or pre-formed and spotted by contact or ink-jet technology (as commercially provided by Agilent or Applied Biosystems). It will be apparent to those skilled in the art that whole or partial cDNA sequences may also serve as probes for array technology (as commercially provided by Clontech). Oligonucleotide probes may be used in blotting techniques, such as Southern blotting or northern blotting, to detect and compare gene expression (for example by means of cDNA or mRNA target molecules representative of gene expression). Techniques and reagents suitable for use in Southern or northern blotting techniques will be well known to those of skill in the art. Briefly, samples comprising DNA (in the case of Southern blotting) or RNA (in the case of northern blotting) target molecules are separated according to their ability to penetrate a gel of a material such as acrylamide or agarose. Penetration of the gel may be driven by capillary action or by the activity of an electrical field. Once separation of the target molecules has been achieved these molecules are transferred to a thin membrane (typically nylon or nitrocellulose) before being immobilized on the membrane (for example by baking or by ultraviolet radiation). Gene expression may then be detected and compared by hybridisation of oligonucleotide probes to the target molecules bound to the membrane.

In certain circumstances the use of traditional hybridisation protocols for comparing gene expression may prove problematic. For example blotting techniques may have difficulty distinguishing between two or more gene products of approximately the same molecular weight since such similarly sized products are difficult to separate using gels. Accordingly, in such circumstances it may be preferred to compare gene expression using alternative techniques, such as those described below.

Gene expression in a sample representing gene expression in a subject may be assessed with reference to global transcript levels within suitable nucleic acid samples by means of high-density oligonucleotide array technology. Such technologies make use of arrays in which oligonucleotide probes are tethered, for example by covalent attachment, to a solid support. These arrays of oligonucleotide probes immobilized on solid supports represent preferred components to be used in the methods and kits of the invention for the comparison of gene expression. Large numbers of such probes may be attached in this manner to provide arrays suitable for the comparison of expression of large numbers of genes selected from those listed above and in Table 2. Accordingly it will be recognised that such oligonucleotide arrays may be particularly preferred in embodiments of the methods of the invention where it is desired to compare expression of more than one gene of the invention. In another embodiment, RNA-Seq (RNA sequencing), also called whole transcriptome shotgun sequencing (WTSS), which relies on next-generation sequencing (NGS) to reveal the presence and quantity of RNA in a biological sample at a given moment in time, is used to compare expression of genes.

Other suitable methodologies that may be used in the comparison of nucleic acid targets representative of gene expression include, but are not limited to, nucleic acid sequence based amplification (NASBA); or rolling circle DNA amplification (RCA).

In some embodiments, said inhibitory compound is a small molecule, an antibody or a siRNA. In an embodiment, the compound is substantially purified (e.g., substantially free from substances that limit its effect or produce undesired side-effects). The subject is in some embodiments, an animal, including but not limited to animals such as cows, pigs, horses, chickens, cats, dogs, etc., and is in some embodiments, a mammal, for example human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound, e.g., encapsulation in liposomes, microparticles, microcapsules, recombinant cells capable of expressing the compound, receptor-mediated endocytosis (see, e. g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.) In yet another embodiment, the compound or composition can be delivered in a controlled release system. In one embodiment, a pump may be used (see Langer, supra; Sefton, CRC Crit. Ref, Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g. Goodson, in Medical Applications of Controlled Release, supra, vol. 2, pp. 115-13 8 (1984)). Other controlled release systems are discussed in the review by Langer (Science 249:1527-1533 (1990)).

The present invention also provides pharmaceutical compositions for use in the treatment of influenza. Such compositions comprise a therapeutically effective amount of an inhibitory compound, and a pharmaceutically acceptable carrier. In a specific embodiment, the term “pharmaceutically acceptable” means approved by a regulatory agency of the Federal or a state government or listed in the U. S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, tale, sodium chloride, driied skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, in some embodiments, in purified form, together with a suitable amount of carrier so as to provide the form for proper administration to the patient. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lidocaine to ease pain at the site of the injection.

Generally, the ingredients are supplied either separately or mixed together in unit dosage form, for example, as a dry lyophilized powder or water free concentrate in a hermetically scaled container such as an ampoule or sachette indicating the quantity of active agent.

Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms.

Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. The amount of the compound which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patients circumstances.

Effective doses may be extrapolated from dose-response curves derived from in vitro or animal model test systems. For antibodies, the dosage administered to a patient is typically 0.1 mg/kg to 100 mg/kg of the patients body weight. In some embodiments, the dosage administered to a patient is between 0.1 mg/kg and 20 mg/kg of the patients body weight, for example1 mg/kg to 10 mg/kg of the patients body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

Also encompassed is a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The antibodies as encompassed herein may also be chemically modified derivatives which may provide additional advantages such as increased solubility, stability and circulating time of the polypeptide, or decreased immunogenicity (see U.S. Pat. No. 4,179,337). The chemical moieties for derivatisation may be selected from water soluble polymers such as polyethylene glycol, ethylene glycol/propylene glycol copolymers, carboxymethyl cellulose, dextran, polyvinyl alcohol and the like. The antibodies may be modified at random positions within the molecule, or at predetermined positions within the molecule and may include one, two, three or more attached chemical moieties. The polymer may be of any molecular weight, and may be branched or unbranched. For polyethylene glycol, the preferred molecular weight is between about 1 kDa and about 100000 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,600, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa. As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999). The polyethylene glycol molecules (or other chemical moieties) should be attached to the protein with consideration of effects on functional or antigenic domains of the protein. There are a number of attachment methods available to those skilled in the art, e.g., EP 0 401 384 (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group. As suggested above, polyethylene glycol may be attached to proteins via linkage to any of a number of amino acid residues. For example, polyethylene glycol can be linked to proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein. As indicated above, pegylation of the proteins of the invention may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. Nos. 4,002,531; 5,349,052; WO 95/06058; and WO 98/32466.

The antagonist of HDAC6 may be contained within compositions having a number of different forms depending, in particular on the manner in which the composition is to be used. Thus, for example, the composition may be in the form of a capsule, liquid, ointment, cream, gel, hydrogel, aerosol, spray, micelle, transdermal patch, liposome or any other suitable form that may be administered to a person or animal. It will be appreciated that the vehicle of the composition of the invention should be one which is well tolerated by the subject to whom it is given, and in some embodiments, enables delivery of the inhibitor to the target site.

The antagonist of HDAC6 may be used in a number of ways.

For instance, systemic administration may be required in which case the compound may be contained within a composition that may, for example, be administered by injection into the blood stream.

Injections may be intravenous (bolus or infusion), subcutaneous, intramuscular or a direct injection into the target tissue (e.g. an intraventricular injection-when used in the brain). The inhibitors may also be administered by inhalation (e.g. intranasally) or even orally (if appropriate).

The inhibitors of the invention may also be incorporated within a slow or delayed release device. Such devices may, for example, be inserted in the body of the subject, and the molecule may be released over weeks or months. Such devices may be particularly advantageous when long term treatment with an antagonist of HDAC6 is required and which would normally require frequent administration (e.g. at least daily injection).

It will be appreciated that the amount of an inhibitor that is required is determined by its biological activity and bioavailability which in turn depends on the mode of administration, the physicochemical properties of the molecule employed and whether it is being used as a monotherapy or in a combined therapy. The frequency of administration will also be influenced by the above-mentioned factors and particularly the half-life of the inhibitor within the subject being treated.

Optimal dosages to be administered may be determined by those skilled in the art, and will vary with the particular inhibitor in use, the strength of the preparation, and the mode of administration. Additional factors depending on the particular subject being treated will result in a need to adjust dosages, including subject age, weight, gender, diet, and time of administration.

When the inhibitor is a nucleic acid conventional molecular biology techniques (vector transfer, liposome transfer, ballistic bombardment etc) may be used to deliver the inhibitor to the target tissue. Known procedures, such as those conventionally employed by the pharmaceutical industry (e.g. in vivo experimentation, clinical trials, etc.), may be used to establish specific formulations for use according to the invention and precise therapeutic regimes (such as daily doses of the gene silencing molecule and the frequency of administration).

Generally, a daily dose of between 0.01 μg/kg of body weight and 0.5 g/kg of body weight of an antagonist of HDAC6 may be used for the treatment of influenza infections in the subject, depending upon which specific inhibitor is used. When the inhibitor is an siRNA molecule, the daily dose may be between 1 pg/kg of body weight and 100 mg/kg of body weight, in some embodiments, between approximately 10 pg/kg and 10 mg/kg, or between about 50 pg/kg and 1 mg/kg.

When the inhibitor (e.g. siNA) is delivered to a cell, daily doses may be given as a single administration (e.g. a single daily injection).

Various assays are known in the art to test dsRNA for its ability to mediate RNAi (see for instance Elbashir et al., Methods 26 (2002), 199-213). The effect of the dsRNA according to the present invention on gene expression will typically result in expression of the target gene being inhibited by at least 10%, 33%, 50%, 90%, 95% or 99% when compared to a cell not treated with the RNA molecules according to the present invention.

Similarly, various assays are well-known in the art to test antibodies for their ability to inhibit the biological activity of their specific targets. The effect of the use of an antibody according to the present invention will typically result in biological activity of their specific target being inhibited by at least 10%, 33%, 50%, 90%, 95% or 99% when compared to a control not treated with the antibody.

The term inhibiting or antagonist (compound/agent) as used herein refers to a molecule which, when binding or interacting with a protein, or with functional fragments thereof, decreases the intensity or extends the duration of the biological activity of said protein. This definition further includes those compounds that decrease the expression of the gene coding for said protein. An inhibiting agent may be made up of a peptide, a protein, a nucleic acid, carbohydrates, an antibody, a chemical compound or any other type of molecule decreasing the effect and/or the duration of the activity of the target protein.

HDAC6, also known as histone deacetylase 6, EC 3.5.1.983, HD6, JM211, FLJ16239, OTTHUMP00000032398, KIAA0901, or OTTHUMP00000197663, plays a central role in microtubule-dependent cell motility via deacetylation of tubulin and cortactin. In addition to its protein deacetylase activity, HDAC6 binds with high affinity ubiquitin or ubiquitin chains and plays a key role in the degradation of misfolded proteins, i.e. when misfolded proteins are too abundant to be degraded by the chaperone refolding system and the ubiquitin-proteasome, HDAC6 mediates the transport of misfolded proteins to the aggresome, a cytoplasmic juxtanuclear structure and also promotes the formation of stress granules. HDAC6 belongs to class IIb of the histone deacetylase/acuc/apha family. It contains an internal duplication of two catalytic domains which appear to function independently of each other. Although it is mostly cytoplasmic, this protein possesses histone deacetylase activity and can repress transcription if present in the nucleus. Additional known substrates of HDAC6 are the chaperone Hsp90 or the actin-binding protein cortactin. In some experiments HDAC6 has been shown to deacetylate the N-terminal tails of histones. Histone deacetylation gives a tag for epigenetic repression and plays an important role in transcriptional regulation, cell cycle progression and developmental events. Analysis of the residues necessary for catalytic activity of HDAC6 has been based mostly on monitoring deacetylation of alpha-tubulin, and revealed that CD2 is active, while CD1 has no activity. Based on these findings, CD1 has been considered to be inactive on physiological substrates, although activity was seen on small peptidic substrates (Hai & Christianson, Nat Chem Biol, 2016; Miyake et al., Nat Chem Biol, 2016). Development of HDAC6 inhibitors has focused on inhibiting CD2 and ignored the potential of CD1. The inventors now made the observation that CD1 is able to deacetylate a novel class of physiological substrates that contain IDRs. Since IDRs are at the basis of liquid-liquid phase separation and the formation of aggregates, developing inhibitors specific for CD1 and/or CD2 will allow to separate the effects of HDAC6 on aggregation vs tubulin deacetylation.

The HDAC6 gene is expressed relatively ubiquitously and is not known to be induced in response to stimuli. It has been shown that acetylation of HDAC6 by p300 attenuates its deacetylase activity (Han Y et al., 2009). Also, Aurora kinase A (AurA) colocalizes with HDAC6 at the basal body of cilia and phosphorylates it, thereby enhancing its tubulin deacetylase activity (Pugacheva et al., 2007). Furthermore, it was also shown that protein kinase CKII phosphorylates HDAC6 on Serine 458, increasing its deacetylase activity and promoting formation and clearance of aggresomes (Watabe and Nakaki, 2012).

As used herein, the “enzymatic activity of HDAC6” refers to the enzymatic (deacetylase) activity of HDAC6, whereas the capacity of HDAC6 to bind ubiquitinated proteins is referred to as “ubiquitin-binding activity of HDAC6” or “ubiquitin-binding property of HDAC6”.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Examples

### DNA Constructs and Antibodies

DDX3X cDNA was cloned from WT MEF cells into expression vectors (pcDNA3.1 or pMSCV). Expression vectors for HATs (FLAG-PCAF, HA-CBP, Myc-Tip60 and HA-p300) were kindly supplied from Dr. Renate Voit (German Cancer Research Center, Heidelberg). To construct expression vectors for functional mutants of HDAC6 and DDX3X, a plasmid of interest was amplified with appropriate sets of primers, then the PCR product was self-ligated to obtain a mutated plasmid. Primary antibodies were as follows: anti-α-tubulin (DM1A) (Sigma, T9026), anti-Acetylated Tubulin (6-11B-1) (Sigma, T7451), anti-FLAG (M2) (Sigma, F1804), anti-Histone H3 (abcam (ab) 1791), anti-HDAC6 (ab56926), anti-HA (16612) (ab130275), anti-c-Myc (9E10) (ab206485), anti-Phopho-eIF2a (Ser51) (D9G8) (Cell Signaling Technology (CST) #3398), anti-ubiquitin (P4D1) (CST #3936), anti-Histone H3 (D1H2) (CST #4499), anti-PABP1 (CST #4992), anti-eIF2a (D7D3) (CST #5324), anti-HDAC6 (D21B10) (CST #7612), anti-DDX3 (D1964) (CST #8192), anti-TIAR (D32D3) (CST #8509), anti-Acetylated-Lysine (CST #9441), anti-Acetylated-Lysine (Ac-K-103) (CST #9681), anti-Acetylated-Lysine (Ac-K2-100) (CST #9814), anti-PABP1 (clone 10E10) (Millipore #04-1467), anti-acetyl-Histone H3 (Millipore #06-599), anti-DDX3X (Millipore #09-860), anti-Acetylated-Lysine (4G12) (Millipore #05-515), anti-Acetylated-Lysine (Immunechem, ICP0380), anti-Acetylated-Lysine (106) (Thermo Fisher Scientific, MA1-2021), anti-DDX3X (clone 15D1 B11) (BioLegend #658602), anti-[K(Ac)40]-α-tubulin (Enzo) and anti-G3BP (Aviva Systems Biology). Anti-mouse-HDAC6 was developed in Matthias laboratory, FMI. Secondary antibodies were as follows: Amersham ECL Mouse IgG, HRP-linked whole Ab from sheep (GE Healthcare) and Amersham ECL Rabbit IgG, HRP-linked whole Ab from donkey (GE Healthcare), Alexa Fluor 488 Goat anti-Rabbit IgG (H+L) Secondary Antibody (invitrogen) and Alexa Fluor 568 Goat anti-Mouse IgG (H+L) Secondary Antibody (invitrogen).

### Establishment and Characterization of K118 Acetylated DDX3X Specific Antibody

The DDX3X-K118 acetylated peptide, CDRSGFGK(Ac)FERG (PSL Peptide Speciality Laboratories) was conjugated with mcKLH by Imject Maleimide Activated Carrier Protein Spin Kits (Thermo scientific) and the KLH conjugated peptide was used to immunize two rabbits (Pocono Rabbit Farm & Laboratory). Collected serum was passed over K118 acetylated peptide CDRSGFGK(Ac)FERG-conjugated agarose column prepared by SulfoLink Immobilization Kit for Peptides (Thermo scientific) and eluted with 0.2 M glycine-HCl, pH 2.0 and neutralized. Then, the elution was passed over K118 unacetylated peptide CDRSGFGKFERG-conjugated agarose column and the flowthrough was used as K118 acetylated DDX3X specific antibody. The specificity of AcK118 was assessed by ELISA using K118 acetylated and unacetylated peptide-coated plates (TaKaRa) and 1-Step Slow TMB-ELISA (Thermo scientific).

### Cell Culture

MEF cells (WT and HDAC6 KO), HEK293T cells and Plat-E packaging cells were cultured in DMEM supplemented with 10% heat-inactivated FBS, penicillin, and streptomycin at 37° C. and 5% CO₂.

### Transfection

HEK293T cells were transfected with Lipofectamine 2000 (Life Technologies) or FuGENE HD (Promega), and MEF cells were transfected with 4D-Nucleofector™ System (Lonza), following manufacturer's protocol.

### Immunoblotting and Immunoprecipitation

Cells were washed by ice-cold PBS and lysed in Triton lysis buffer (50 mM Tris-HCl, pH8.0, 150 mM sodium chloride, 1 mM EDTA, 0.1% TritonX-100 and Complete EDTA-free protease inhibitors (Roche)) for analysis. To detect protein acetylation, 0.2 μM Trichostatin A and 5 mM nicotinamide was added to PBS for wash, and 10 μM Trichostatin A, 10 mM nicotinamide, 50 mM sodium butyrate were added to Triton lysis buffer. To detect protein phosphorylation, Pierce Protease and Phosphatase Inhibitor Mini Tablets (Thermo scientific) were added to Triton lysis buffer instead of Complete EDTA-free protease inhibitors. Five hundred μg protein lysates were incubated with the specific antibody for overnight at 4° C., and immunoprecipitated with Dynabeads ProteinG (invitrogen) for 1 h at 4° C. Samples were boiled for 10 min in SDS-PAGE sample buffer, and separated with 4-12% NuPAGE gels (invitrogen). Proteins were transferred onto PVDF membranes (Immobilon-P, Millipore), proved with specific primary antibody for overnight and secondary antibody for 1 h under 5% non-fat dry milk in TBS blocking condition and detected with Amersham ECL Western blotting reagent (GE Healthcare). For inhibition of HDACs in cells, concentration of each HDAC inhibitor was as described in {Scholz, 2015 #851}.

### Biotinylated Isoxazole (B-Isox)-Mediated Precipitation

Cells were washed by ice-cold PBS and lysed in EE buffer (50 mM HEPES, pH7.5, 150 mM sodium chloride, 1 mM EDTA, 2.5 mM EGTA, 0.1% NP-40, 10% Glycerol, 1 μM DTT and Complete EDTA-free protease inhibitors (Roche)) To detect DDX3X acetylation, 10 μM Tubacin was added to EE buffer. Biotinylated isoxazole (Sigma T511617) in DMSO was added to cell lysates at 100 μM final concentration. The reaction solutions were incubated for 4 hr at 4° C. and then centrifuged at 13200 rpm for 10 min. Precipitates were washed five times in EE buffer before SDS solubilization, following analyzed by western blotting as mentioned above.

### Immunofluorescence Microscopy

Cells on a Poly-D-lysine coated coverglass (neuVitro) were washed by ice-cold PBS, then fixed with ice-cold methanol or 4% paraformaldehyde. After permeabilization with 0.5% TritonX-100 in PBS for 10 min, the cells were incubated with specific primary antibody for overnight and secondary antibody for 1 h, then mounted with ProLong Gold Antifade Reagent (Cell Signaling Technology #9071). Images were captured by Axioimager Z1 microscope (Zeiss).

### Establishment of KO Cell Lines Using CRISPR/Cas9 Genome Editing

HDAC6 KO 293T cells were established by CRISPR/Cas9 genome editing. The guide sequence targeting human HDAC6 was designed by the CRISPR design tool at http://www.genome-engineering.org/crispr/ and cloned into pX330-Cas9-T2A-mCherry vector (Addgene).

The pX330 vector was transfected into HEK293T cells as mentioned above. Two days after transfection, the mCherry positive cells were collected by FACS. Then single cell clones were sorted again week after and expanded. Screening for HDAC6 knockout was done by western blotting. Genomic DNA was purified from the HDAC6 KO clone and the region surrounding PAM of the sgRNA was cloned into pGEM-T Easy vector (Promega) for sequencing after amplification with a pair of primers.

To determine the indels of individual alleles, the amplicons from 20 bacterial colonies were sequenced. Establishment of DDX3X KO MEFs followed the same procedure.

### In Vitro HDAC6 Deacetylation Assay

Danio rerio HDAC6 CD1-CD2 (a.a.40-831) was expressed in Sf9 insect cells and purified based on the method previously described {Miyake, 2016 #1420}. Two μg of the DDX3X-K118 acetylated peptide, DRSGFGK(Ac)FERG was reacted for 3 hours with 10 pmol purified HDAC6 CD1-CD2 in 20 μl of HDAC Assay Buffer (50 mM Tris-HCl, pH8.0, 137 mM sodium chloride, 2.7 mM potassium chloride, 1 mM magnesium chloride, a component of HDAC assay kit from Enzo) at 37° C. and the reaction was terminated with 1.0% trifluoroacetic acid. The peptide and HDAC6 CD1-CD2 were also reacted under 1 μM Trichostatin A. The amount of acetylated and deacetylated peptides was calculated, following the peptide separation by High-performance liquid chromatography and measuring the UV at 214 nm.

### Computational Prediction of Disordered Regions

Computational prediction of disordered regions was done with the PONDR VSL2 program {Peng, 2006 #63}.

### HDAC6-Dependent Acetylome in MEFs Identifies DDX3X

To identify novel substrates of HDAC6, the inventors utilized mass spectrometry data to uncover acetylome changes upon chemical inhibition or genetic ablation of HDAC6 activity. For this, the inventors re-analyzed mouse embryonic fibroblasts (MEFs) acetylome datasets {Scholz, 2015 #851}, comparing data obtained with two different HDAC6-specific inhibitors (Tubacin or Bufexamac) to those obtained with HDAC6 KO MEFs. They plotted the SILAC ratios in two dimensions (inhibitor vs control, or KO vs WT,): about 4% of the quantified sites (87 out of 2029) displayed a more than 2-fold increase in acetylation in either of these three conditions (Tubacin, Bufexamac or HDAC6 KO). Gene ontology and STRING {Szklarczyk, 2017 #1486} analysis of the 87 sites whose acetylation is upregulated in either of the three conditions identified proteins related to cytoskeleton, trafficking, RNA metabolism, translation control, mitochondrial function as well as mitosis. Moreover, about 1% of the sites (18 out of 2029) showed a more than 2-fold increase in acetylation in all three conditions. Hereafter, the inventors refer to these 18 sites as “high confidence HDAC6 target sites”. Within this group, three sites are in the well-known HDAC6 substrate cortactin (Cttn), demonstrating the validity of the method. Analysis of these high confidence HDAC6 target sites with IceLogo {Colaert, 2009 #1459} defined a loose consensus HDAC6 substrate site where several positions show an enrichment, in particular the two residues immediately before the acetylated lysine: -GGK(Ac)-.

Among the high confidence HDAC6 target sites, the DDX3X-K118 acetylation (SGFGK(Ac)FER) caught the inventor's interest because it showed one of the highest SILAC ratios in the datasets used, was recently identified in an acetylome study of mouse liver {Zhang, 2015 #878}, and offers a possible link between HDAC6 and the stress response. DDX3X is a DEAD box helicase, whose main function is to unwind duplex RNA using ATP and remodel RNA-protein complexes {Linder, 2011 #883}. HDAC6 and DDX3X were both independently reported to be localized to SGs under oxidative stress caused by arsenite {Shih, 2012 #387; Kwon, 2007 #168; Legros, 2011 #1328}, suggesting that they may physically interact. To confirm the data from the acetylome analysis, the inventors immunoprecipitated DDX3X from HDAC6 KO MEFs lysates using an acetylated-lysine antibody and verified acetylation of K118 by mass spectrometry analysis. These results reveal that DDX3X is a lysine-acetylated protein and that its acetylation level is increased in absence of HDAC6.

### DDX3X is a Novel HDAC6-Specific Substrate

To investigate whether DDX3X-K118Ac is a direct target site of HDAC6, the inventors performed a biochemical assay using purified components. They found that a purified protein consisting of the tandem HDAC6 catalytic domains {Miyake, 2016 #1420} efficiently deacetylated the DDX3X-K118Ac peptide (SGFGK(Ac)FER) in vitro, and this reaction was inhibited by the pan-HDAC inhibitor Trichostatin A (TSA). Next, they tested the acetylation status of DDX3X in cells. It has been shown that the stoichiometry of acetylation is generally low {Weinert, 2014 #452}, making it difficult to detect. The inventors therefore attempted to enhance the level of acetylation by expressing different HATs. To test which HAT(s) can act on DDX3X, expression vectors for PCAF, CBP, Tip60 or p300 were individually transfected into HEK293T cells and cellular extracts were analyzed by immunoprecipitation and immunoblotting. Both CBP and p300 led to detectable acetylation of endogenous DDX3X, whereas Tip60 and PCAF did not. Further, they detected robust acetylation of DDX3X when cells were transiently co-transfected with constructs encoding CBP or p300 together with a construct for DDX3X. The acetylation level of endogenous or overexpressed DDX3X was increased even further following treatment of the cells with Tubacin.

To confirm that the DDX3X-K118Ac is the target site of HDAC6, the inventors co-expressed an acetylation-dead DDX3X-K118R mutant (a mutant that cannot be acetylated) with CBP in HEK293T cells. The acetylation signal was strongly decreased in DDX3X-K118R compared to DDX3X3-WT, indicating that the signal in these immunoblotting experiments arises primarily from K118Ac. Next, to detect DDX3X-K118Ac more specifically, the inventors generated a DDX3X-K118Ac-specific antibody which was affinity-purified and showed high specificity. The inventors established HDAC6 KO HEK293T cells by CRISPR/Cas9 genome editing and observed increased acetylation of DDX3X in HDAC6 KO compared to parental HEK293T cells with this DDX3X-K118Ac-specific antibody, further confirming that DDX3X-K118 is a target of HDAC6.

Different deacetylases often redundantly target the same lysine acetylation site on a target protein. For example, the two established HDAC6 substrates α-tubulin and cortactin have also been reported as substrates of SIRT2, a class III deacetylase {North, 2003 #1386; Zhang, 2007 #174}. To test whether the DDX3X-K118 deacetylation is specific for HDAC6, the inventors transiently expressed CBP in HEK293T cells and assessed the DDX3X-K118Ac signal after treating the cells with several HDAC inhibitors. As expected from the acetylome analysis, DDX3X-K118Ac increased upon treatment with the HDAC6 specific inhibitors, Bufexamac and Tubacin. Moreover, Tubastatin A, another HDAC6 specific inhibitor, also increased DDX3X-K118Ac, further supporting the notion that DDX3X-K118 is a HDAC6 target site. In contrast, neither nicotinamide, a sirtuin deacetylase inhibitor, nor sodium butyrate, a class I HDAC inhibitor, altered the acetylation status of DDX3X-K118. These effects of HDAC inhibitors are also observed in an independent HeLa cells acetylome dataset {Scholz, 2015 #851}. Together, these findings indicate that DDX3X is a novel HDAC6-specific substrate.

DDX3X Co-Localizes and Interacts with HDAC6

Both DDX3X and HDAC6 were independently reported to be localized on SGs under oxidative stress {Shih, 2012 #387; Kwon, 2007 #168; Legros, 2011 #1328}. The inventors observed that DDX3X co-localized with the SG component PABP1 under arsenite-induced oxidative stress in HEK293T cells. In agreement with previous observations {Shih, 2012 #387}, they found that the helicase activity of DDX3X is not required for its localization to SGs, as two mutants that inactivate its ATPase activity still co-localize with SGs like the WT protein. Moreover, endogenous DDX3X and HDAC6 both showed a diffuse distribution in the cytosol and co-localized on SGs upon treatment with arsenite in HEK293T cells. Further, overexpressed and endogenous DDX3X both co-immunoprecipitated with HDAC6, irrespective of arsenite treatment.

To define the domains required for interaction between HDAC6 and DDX3X, the inventors transiently expressed full-length or truncated DDX3X proteins in HEK293T cells together with full-length HDAC6, and performed co-immunoprecipitation assays. Truncated DDX3X mutants lacking the N-terminal region (“del-Nt” and “Ct” mutants) were poorly expressed, suggesting that the N-terminal region is important for protein stability; however, all other constructs were well-expressed. The full-length DDX3X and del-Helicase mutants co-immunoprecipitated efficiently with HDAC6. In contrast, truncated mutants lacking the C-terminal region (“del-Ct” and “Nt” mutants) were not co-immunoprecipitated with HDAC6, although they were stably expressed. These results indicate that the C-terminal region of DDX3X is important for its interaction with HDAC6. To determine if this region can confer HDAC6 binding activity on another DEAD box family protein, the inventors replaced the C-terminal region of the DEAD box family protein DDX5 with that of DDX3X (DDX5+3XCt) and performed similar co-immunoprecipitation assays. They found that the DDX5+3XCt chimeric protein robustly co-immunoprecipitated with HDAC6, whereas the control DDX5 did not. Thus, the DDX3X C-terminal region is necessary and sufficient to interact with HDAC6.

### DDX3X-K118 Acetylation is Induced by Oxidative Stress Independently of Stress Granules

It was recently reported that TDP-43, an RNA binding protein linked to amyotrophic lateral sclerosis (ALS), is a substrate of HDAC6 and that acetylation of its RNA binding domain, which is increased by oxidative stress, leads to formation of TDP-43 aggregates {Cohen, 2015 #907}. Although DDX3X does not have an RNA binding domain, its acetylation site K118 is located in the N-terminal region of DDX3X, which is defined as an intrinsically disordered region (IDR) by PONDR analysis.

Posttranslational modifications are thought to alter the propensity of IDRs to aggregate, as exemplified by the phosphorylation of the FUS IDR {Han, 2012 #1389}. Given their findings and these previous observations, the inventors hypothesized that acetylation/deacetylation of DDX3X by HDAC6 may alter the properties of its IDR and modulate its capacity to form SGs.

First, they determined whether oxidative stress caused by sodium arsenite alters the acetylation status of DDX3X. They found that DDX3X-K118 acetylation was increased after arsenite treatment of WT cells and was further increased when cells were also treated with Tubacin, or when they lacked HDAC6. It was previously shown that residues 38-44 in DDX3X are important for binding to the translation initiation factor eIF4E and that this interaction is critical for inducing SG formation {Shih, 2012 #387}. To determine if DDX3X-K118 acetylation during oxidative stress is influenced by SG formation, the inventors compared the acetylation status of two DDX3X eIF4E-binding-defective mutants, Y38A and L43A, to that of the WT protein. Transiently expressed DDX3X Y38A and L43A formed significantly less SGs than WT in arsenite-treated cells, but their acetylation status was the same as that of WT DDX3X. Thus, acetylation of DDX3X-K118 in response to oxidative stress appears to be largely independent of localization to SGs.

### HDAC6 Promotes Stress Granule Maturation Through DDX3X Deacetylation

To investigate the role of DDX3X-K118 acetylation on SG formation, the inventors individually expressed three DDX3X constructs in WT MEFs: WT, K118R (a mutant that cannot be acetylated), and K118Q (an acetyl-lysine mimic mutant). As expected, the WT protein localized to SGs under oxidative stress caused by arsenite and did not form SGs without stress. The two mutants, K118R which mimics a non-acetylated lysine, and K118Q which mimics an acetylated lysine, also formed SGs in the presence of arsenite; whereas the DDX3X-K118R SGs were similar to that of the WT protein, the K118Q SGs had a clearly different appearance. To further explore the effect of these mutants on SG dynamics, the inventors quantified the number and size of SGs. On average, the number of SGs per cell was significantly increased in the K118Q mutant, but not in the K118R mutant (WT: 18±7, K118Q: 33±13, K118R: 21±9, FIG. 5C). This was accompanied by an overall shift in SG size, as the K118Q mutant formed significantly smaller SGs (WT: 0.75±0.18 μm², K118Q: 0.52±0.14 μm², K118R: 0.69±0.17 μm²). This behavior of K118Q implies that acetylated DDX3X tends to form numerous smaller SGs. In agreement with this, acetylation of endogenous DDX3X induced by CBP also increased the number of SGs in 293T cells. To further interrogate the relationship between DDX3X acetylation and SG formation, the inventors generated DDX3X KO MEFs by CRISPR/Cas9 genome editing and established rescue lines re-expressing either WT or DDX3X-K118R. Tubacin treatment of the DDX3X WT rescue line reduced the average size of SGs, while slightly increasing their number. In contrast, in the cells expressing the DDX3X-K118R mutant that cannot be acetylated, the number and size of SGs was not significantly altered by HDAC6 inhibitio. Thus, HDAC6 promotes the formation of mature SGs during oxidative stress via deacetylation of DDX3X-K118.

Acetylated Lysines in IDRs Control their Aggregation Properties and are Regulated by Both CD1 and CD2 Domains of HDAC6

To further examine the role of acetylation on the capacity of DDX3X to assemble in granules, the inventors performed in vitro experiments with the chemical biotinylated isoxazole (B-isox). Previous studies by McKnight and colleagues showed that B-isox forms microcrystals that can selectively precipitate from cellular lysates proteins with low complexity (LC) domains, which are a subset of IDRs with little diversity in their amino acid composition {Han, 2012 #1389}. For simplicity, the inventors make no specific distinction hereafter between IDRs and LCs. This assay has been used to assess the capacity of proteins, such as FUS, to form cellular RNA-containing granules {Han, 2012 #1389}. We expressed CBP or GFP (negative control) in HEK293T cells and found that B-isox robustly precipitated DDX3X and another SG component, TIAR, both of which contain an IDR. Remarkably, only non-acetylated DDX3X was precipitated, demonstrating that the acetylation status of its IDR is critical for the capacity of this protein to form stable aggregates. Ubiquitin and α-tubulin, both of which lack an IDR, were not precipitated by B-isox, thus confirming the specificity of the assay. Taken together, these findings demonstrate that oxidative stress-induced acetylation of DDX3X-K118 in the IDR reduces its propensity to form granules and that HDAC6-mediated deacetylation of DDX3X is required for SGs to mature.

The inventors next examined the high-confidence HDAC6 target sites to determine which fraction of these sites map to an IDR. When all 2029 acetylated sites are examined by PONDR analysis, ca. 50% of them fall in an IDR. In striking contrast, >93% of the high-confidence HDAC6 target sites map in an IDR, suggesting that HDAC6 may have a role to regulate IDRs beyond DDX3X.

Based on the above observations the inventors wondered whether acetylation/deacetylation of IDRs may be a general mechanism to regulate their aggregation properties. To examine this, they used lysates from HDAC6 KO 293T cells for B-isox precipitation experiments and analyzed the different fractions by immunoblotting with a pan-acetyl-lysine antibody. In absence of exogenous CBP expression, only one acetylated protein is detected with an apparent molecular weight of 50 kDa; it corresponds to acetylated alpha-tubulin, whose acetylation level is greatly increased in cells lacking HDAC6 {Miyake, 2016 #23; Zhang, 2008 #66}. Expression of CBP leads to the appearance of multiple additional signals, which are all reduced when HDAC6 is also co-expressed. In striking contrast, the precipitate fraction shows almost no signal under any of the conditions; the robust precipitation of TIAR, which has an IDR, confirmed that the precipitation was effective. This surprising observation suggests that the acetylation signals detected in these experiments are either in proteins that lack an IDR—such as alpha-tubulin-, or are in IDRs and preclude b-isox-mediated precipitation.

Recent crystallographic and functional studies showed that the catalytic activity of HDAC6, as assayed with its main previously known substrate alpha-tubulin, resides the second catalytic domain CD2, whereas CD1 shows activity only with some small peptide or synthetic substrates {Hai, 2016 #49; Miyake, 2016 #1420}. To determine which catalytic domain(s) is responsible for DDX3X-K118Ac deacetylation, the inventors expressed different HDAC6 functional mutants (H216A, CD1 catalytic dead; H611A, CD2 catalytic dead; H216A/H611A double mutant, and W1182A, ubiquitin-binding-deficient) {Hao, 2013 #336; Grozinger, 1999 #929} in HDAC6 KO HEK293T cells together with CBP. Analysis was done by immunoblotting to monitor the acetylation level of different endogenous target proteins, using either an antibody specific for the acetylated protein (Ac-DDX3X-K118 or Ac-alpha-tubulin) or an anti-acetylated lysine antibody (cortactin). The catalytically-inactive H216A/H611A double mutant did not deacetylate any substrate, as anticipated. However, and unexpectedly, each single catalytic domain HDAC6 mutant was able to robustly deacetylate DDX3X and also to a lesser extent cortactin. This indicates that either domain, CD1 or CD2, is active to deacetylate DDX3X or cortactin, two proteins containing an acetylated IDR. This reactivity is different from the reactivity for α-tubulin, which requires the catalytic activity from CD2 to be deacetylated, as was previously known {Haggarty, 2003 #51; Miyake, 2016 #23}.

Finally, an analysis of proteins known to be involved degenerative disease associated with the formation of intracellular granules, showed that many of them display at least one HDAC6 deacetylation site, mostly in IDR.

1. A HDAC6 inhibitor for use in treating a degenerative disease associated with the formation of intracellular granules, wherein said granules are formed by the aggregation of a protein selected from the group comprising APP, Tau, Ataxin-2, hnRNPA1, C9orf72, Lamin A/C, Myotilin, Matrin 3, alpha-synuclein, SOD1, FUS, hnRNPA2B1, VCP, TIA1, Desmin and Huntingtin, and wherein said inhibitor inhibits both the CD1 and CD2 of HDAC6.

2. A method for treating, in a patient in need thereof, a degenerative disease associated with the formation of intracellular granules, wherein said granules are formed by the aggregation of a protein selected from the group comprising APP, Tau, Atx2, hnRNPA1, C9orf72, Lamin A/C, Myitilin, Matrin 3alpha-synuclein, SOD1, FU, hnRNPA2, VCP, TIA1, Desmin and Hungtitin, said method comprising administering a therapeutically effective amount of an inhibitor inhibiting both the CD1 and CD2 of HDAC6.
